(19) United States
(12) Patent Application Publication
(10) Pub. No.: US 2024/0299511 A1
(43) Pub. Date: Sep. 12, 2024

(54) AN IMMUNOGEN

(71) Applicant: UNIVERSITY OF PRETORIA, Pretoria (ZA)

(72) Inventor: Robert Peter MILLAR, Pretoria (ZA)

(21) Appl. No.: 18/273,087

(22) PCT Filed: Jan. 26, 2022
(86) PCT No.: PCT/IB2022/050661

§ 371 Date: Jul. 19, 2023

(30) Foreign Application Priority Data
Jan. 28, 2021 (GB) 2101187.9

(51) Int. Cl. A61K 39/00; A61P 15/16; A61P 15/18; C07K 14/005; C07K 14/475; C07K 14/59

(52) U.S. Cl. CPC A61K 39/0006; A61P 15/16; A61P 15/18; C07K 14/005; C07K 14/475; C07K 14/59; A61K 2039/55566; A61K 2039/6075; C07K 2319/40; C12N 2730/10122

(57) ABSTRACT

This invention relates to an immunogen comprising a gonadotropin releasing hormone (GnRH) peptide sequence, a kisspeptin peptide sequence and a stimulant of raising an immune response, such an immunogen for use in a method to regulate the release of hormones in a vertebrate including modulation of reproductive hormones, to reduce fertility in a vertebrate and to treat hormone-dependent diseases including hormone-dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity and a vaccine formulation comprising the immunogen. The invention also relates to the use of the immunogen in the preparation of a medicament for use in a method to regulate the release of hormones in a vertebrate.

### INTRODUCTION

This invention relates to an immunogen comprising at least two of a gonadotropin releasing hormone (GnRH) peptide sequence, a kisspeptin peptide sequence, a neurokinin B peptide sequence and other upstream regulators of GnRH, such an immunogen for use in a method to regulate the release of hormones in a vertebrate including modulation of reproductive hormones, to reduce fertility in a vertebrate and to treat hormone-dependent diseases including hormone-dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity and a vaccine formulation comprising the immunogen. The invention also relates to the use of the immunogen in the preparation of a medicament for use in a method to regulate the release of hormones in a vertebrate.

### BACKGROUND TO THE INVENTION

Immunocontraception in laboratory rodents, livestock, wildlife and companion animals have utilised two principle antigens; porcine zona pellucida (pZP) proteins and conjugated GnRH analogues. pZP immunocontraception is effective as a contraceptive in a number of species [1-3] but is reversible, requires reimmunisation, and is confined to female contraception. GnRH vaccines on the other hand are effective in both sexes as the antibodies neutralise endogenous GnRH thereby inhibiting activation of pituitary gonadotropes, release of (follicle stimulating hormone) FSH and (luteinizing hormone) LH and downstream gametogenesis and steroidogenesis. A number of GnRH vaccines have been described in the literature and some like Equity, Improvac and Bopriva [4-7] (Registered Trade Marks (RTM)) are marketed commercially for use in livestock. Improvac® was also used successfully to induce anoestrus in mares but the effect was reversible and older mares were found to be more resistant to immunisation and returned to cyclic activity sooner than younger mares [4, 8-10]. Similarly, in stallions, older animals immunised with Improvac recovered fertility at about 6 months following a primary and single booster vaccination [11]. Using the same vaccine in blackbuck and springbok, testosterone concentrations were reduced in young but not in adult rams [12]. In most male species tested, inhibition of reproduction lasted 6 months or less. However, the inventor in collaboration with colleagues has previously been successful in achieving permanent infertility by immunising neonatal sheep with a GnRH ovalbumin immunogen [13]. Vaccination was commenced at 3-4 weeks of age in ram and ewe lambs and a booster was administered at 13-14 weeks of age. Three to four years after immunisation the animals had undetectable anti-GnRH antibody titers but unexpectedly both rams and ewes were hypogonadal with low or undetectable levels of LH and FSH. Ovariectomy of treated ewes did not produce the expected rise in LH and FSH levels suggesting compromise of GnRH secretion. This extraordinarily long-lasting inhibition of reproductive hormones appeared to be developmentally determined and due to the destruction of GnRH neurons as revealed by immunocytochemistry.

Although GnRH is well established as the central regulator of the reproductive hormone cascade, GnRH neurons lack much of the molecular machinery for the metabolic and sex steroid regulation of reproduction—for example sex steroid receptors. This conundrum was resolved with the discovery of a novel hypothalamic neuropeptide, kisspeptin, and its cognate receptor, GPR54 (see reviews [14-19]). The discovery of kisspeptin and its receptor arose from genomic studies in patients with a failure to progress through puberty and in laboratory mice with inactivating mutations in GPR54 [20, 21] or mutations in kisspeptin itself [22]. Kisspeptin neurons express sex steroid hormone receptors and KiSS1 gene expression is regulated by sex steroids as well as by nutritional, stress, inflammatory and metabolic status (see above reviews).

A need therefore exists for immunogens (particularly GnRH immunogens) to convey infertility to 100% of immunised animals preferably without the reversal of infertility over time, and without the need for reimmunisation.

### SUMMARY OF THE INVENTION

In this specification, the following terms have the meanings as set out.
Immunogen: An antigen or any substance that may be specifically bound by a component of the immune system to stimulate an immune response.
Puberty: The period during which adolescents reach sexual maturity and become capable of reproduction.
Pubertally: Of or relating to puberty.
Prepubertally: Before puberty.
Livestock: Farm animals regarded as an asset, including (but not limited to) cattle, horses, sheeps, goats and pigs.
Wildlife: Wild animals collectively including (but not limited to) elephant, lion, unhorned and horned animals such as antelope, impala, springbok and deer.

Since inactivating mutations of the KiSS1 or GPR54 genes give rise to infertility and studies with kisspeptin antagonists demonstrate an inhibition of GnRH secretion [16] and suppression of the ovulatory LH surge [23], it is reasoned by the inventor that vaccination against kisspeptin may be an immunocontraception target. This notion is supported by the observation that administration of kisspeptin antiserum inhibited the ovulatory surge of LH in female rats [24].

In view of the failure of GnRH immunogens to convey infertility to 100% of immunised animals and the spontaneous reversal of infertility over time, and the need for reimmunisation, it is hypothesized that a combination of GnRH and its upstream regulator, kisspeptin, along with a hepatitis B T helper peptide sequence within a single molecule would be a more efficacious immunogen than a single immunogen targeting a single point in the hypothalamic-pituitary-gonadal axis. It is also proposed that permanent sterilisation might be achieved by immunising during particular windows of development as was achieved in the sheep studies (supra) [13]. Lastly, it is considered that a single immunisation at a specific stage of development might be sufficient to elicit permanent immune sterilisation. This application teaches experiments directed at these proposals in male and female rats at different stages of development and shows that a single immunisation with this novel immunogen results in an inhibition of reproductive capacity for at least 300 days in 100% of pubertal female rats.

GnRH immunogens have been extensively employed in immunocontraception of animals. While they are effective, they are not 100% efficacious and of limited duration. GnRH secretion is dependent on upstream stimulation by kisspeptin. A dual immunogen combining GnRH and kisspeptin was therefore proposed by the inventor to more efficacious through targeting two levels of the hypothalamic/pituitary axis. It has been previously shown GnRH immunogen elicits permanent sterilisation when sheep are vaccinated neonatally suggesting that the efficacy of GnRH immunisation may be dependent on the stage of reproductive development. The present invention teaches, now studied over 300 days, the efficacy of immunisation with a dual immunogen comprising GnRH linked to kisspeptin via a Hepatitis B T helper peptide sequence (GKT) administered to male and female rats prepubertally, pubertally and as adults. At all stages of development all immunised animals produced antibodies to GnRH, kisspeptin and GKT but differentially in titre with respect to sex and stage of development. In immunised adult, prepubertal and pubertal males, testosterone and testes length was markedly reduced by 60 days and remained at low levels until day 150. Thereafter, testosterone recovered to pre immunisation levels and testes length recovered to a maximum of about 40% of controls. 80% of males were infertile in three matings over 250 days. In prepubertal and pubertal female rats a single GKT immunisation at day 0 reduced estradiol to low levels by day 60 which remained low until termination of the experiment on day 300. In matings of these females with fertile males on days 90, 120 and 250, 74% of prepubertal females were infertile and impressively, 100% (10/10) of pubertal females were infertile after a single immunisation on day 0. These findings set the scene for exploration of immunosterilisation of wild and domestic animals after a single immunisation with GKT.

### SPECIFIC DESCRIPTION OF THE INVENTION

Accordingly, in a first aspect to the present invention there is provided an immunogen comprising a gonadotropin releasing hormone (GnRH) peptide sequence, a kisspeptin peptide sequence and a stimulant of raising an immune response including T helper peptide sequences as in tetanus toxin, Hepatitis B and from other polypeptides.

The T helper peptide sequence may be a Hepatitis B peptide sequence. The Hepatitis B peptide sequence preferably links the GnRH and the kisspeptin peptide sequences.

The GnRH peptide sequence may have at least 80% homology with SEQ ID No.1 E-H-W-S-Y-G-L-R-P-G, including 85, 90, 95 and 100% homology.

The kisspeptin peptide sequence may have at least 80% homology with SEQ ID No.2 Y-N-W-N-S-F-G-L-R-F, including 85, 90, 95 and 100% homology.

The T helper peptide sequence may have at least 80% homology with SEQ ID No.3 F-F-L-L-T-R-I-L-T-I-P-Q-S-L-D, including 85, 90, 95 and 100% homology.

In one embodiment of this aspect to the invention the immunogen is a single peptide molecule.

In one embodiment of this aspect to the invention the amino and carboxyl termini are extended or blocked with peptide sequences or addition of peptides or other molecules via central residues including conjugation to immunoglobulin.

It will be appreciated that the order of the peptide sequences may be in any order, including kisspeptin, Hepatitis B T helper sequence and GnRH.

The immunogen may have a peptide sequence of at least 80% homology with SEQ ID No.4 Y-N-W-N-S-F-G-L-R-F-G-F-F-L-L-T-R-I-L-T-I-P-Q-S-L-D-G-E-H-W-S-Y-G-L-R-P-G, including 85, 90, 95 and 100% homology.

In one embodiment of this aspect to the invention the immunogen has a peptide sequence Ac-Y-N-W-N-S-F-G-L-R-F-G-F-F-L-L-T-R-I-L-T-I-P-Q-S-L-D-G-E-H-W-S-Y-G-L-R-P-G-NH2. (SEQ ID No.4)

According to a second aspect to the present invention there is provided an immunogen according to the first aspect of the invention for use in a method to regulate the release of hormones in a vertebrate including modulation of reproductive hormones, to reduce fertility in a vertebrate and to treat hormone-dependent diseases including hormone-dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity, the method comprising the steps of administering the immunogen to a vertebrate in an amount effective to regulate the release of hormones in the vertebrate.

The immunogen may be administered to the vertebrate prepubertally, pubertally and/or as an adult, preferably pubertally.

The immunogen components may be singly administered and the immunogen may be administered intravenously, orally or by subcutaneous injection. Singly administered is considered to include a single injection or other form of administration where the immunogen components are included in the single administration, be the components separate or joined.

According to a third aspect to the present invention there is provided a vaccine formulation comprising an immunogen according to the first aspect to the present invention in an amount effective to regulate the release of hormones in a vertebrate including modulation of reproductive hormones, to reduce fertility in a vertebrate and to treat hormone-dependent diseases including hormone dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity, in combination with a pharmaceutically acceptable carrier or excipient.

In the vaccine formulation the peptide may be conjugated to or administered with at least one carrier or adjuvant including CpGs, M59, incomplete Freund's adjuvant, complete Freund's adjuvant, alum, bile salts, vitamins, PEG, molecules which prolong half-life and attenuated toxins.

The vertebrate may be a mammal selected from humans, rodents, including rats and mice, cats, dogs, livestock including cattle, horses and wildlife.

According to a fourth aspect to the present invention there is provided the use of an immunogen according to the first aspect to the present invention in the preparation of a medicament for use in a method to regulate the release of hormones in a vertebrate including modulation of reproductive hormones, to reduce fertility in a vertebrate and to treat hormone dependent diseases including hormone dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity.

According to further aspects to the present invention there is provided a nucleic acid which encodes the immunogen according to the first aspect to the present invention, an expression vector comprising the nucleic acid and a host cell comprising the expression vector.

### BRIEF DESCRIPTION OF THE FIGURES

FIG. 1. Antibody titres of Anti GnRH, anti kisspeptin and anti GKT in adult male rats immunised with GKT peptide. All animals received four immunisations in a fortnightly schedule at days 0, 15, 30 and 45. Titres were measured at day 60. For the comparison of the titres a non-parametric Kruskal Wallis test and a multiple comparison of Dunn were used. Significant differences of titres are indicated; *(p<0.05); **(p<0.01) and ***(p<0.001) compared with placebo titres. Horizontal lines show the mean value.

FIG. 2. Testosterone levels in adult male rats immunised with the GKT peptide on four occasions as in FIG. 1. (A) placebo animals (B) immunised males (C) Box and whiskers graph representation of testosterone means. For comparison, a two-way ANOVA and Student-Newman-Keuls post hoc test was used. Statistical differences between immunised and placebo animals at different times are denoted. *(p<0.05); **(p<0.001). Each line is an individual animal.

FIG. 3. Length of the testes in adult male rats immunised with GKT (triangles) and in placebo animals (squares). The testes length measurement commenced after the animals completed the four immunisations fortnightly schedule. For comparison, a simple ANOVA and a Tukey posttest were used. Significant differences in testis length between immunised animals and placebo animals are indicated; *(p<0.05); **(p<0.01) and ***(p<0.001) Horizontal lines show the mean value. Symbols on the x axis indicate the testes were unmeasurable.

FIG. 4. Anti-GnRH, anti-kisspeptin and anti-GKT antibody titres in (A) prepubertal males and (B) pubertal males immunised with GKT. For the comparison of antibody titres a non-parametric test of Kruskal Wallis and a multiple comparison of Dunn were used. Titres were measured at day 60 after a single immunisation. Significant differences among immunised animals and placebo are indicated; *(p<0.05); **(p<0.01) and ***(p<0.001). Horizontal lines show the mean value.

FIG. 5. Testosterone levels in prepubertal male rats. (A) testosterone levels of placebo animals. (B) testosterone levels in GKT immunised male rats. Animals received a single immunisation at day 0 and a re-immunisation at day 180 after testosterone levels began to recover. (C) Box and whiskers graph representation of testosterone mean values of placebo and immunised animals. For comparison, a two-way ANOVA and Student-Newman-Keuls post hoc test was used. Statistical differences between immunised and placebo animals at different times are indicated. *(p<0.05).

FIG. 6. Testosterone levels in pubertal male rats. (A) testosterone levels of the placebo males. B) testosterone levels in GKT immunised males. Animals received a single immunisation at day 0 and a re-immunisation at day 180 after testosterone levels began to recover. (C) Box and whiskers graph representation of testosterone mean values of placebo and immunised animals. For comparison, a two-way ANOVA and Student-Newman-Keuls post hoc test was used. Statistical differences between groups at different times are indicated. *(p<0.05).

FIG. 7A. Length of the testes in prepubertal male rats injected (immunised) with placebo (squares) and immunised with the GKT (triangles). The arrows indicate times of immunisation. Horizontal lines show the mean value. Symbols on the x axis indicate the testes were too small to measure. B. Length of the testes in pubertal rats injected with placebo (squares) and immunised with the GKT peptide (triangles). The arrows indicate times of immunisation. For comparison, a Kruskal Wallis test followed by Dunn's multiple comparison was used in both cases. Statistical differences in immunised and placebo animals are indicated; *(p<0.05); **(p<0.01) and ***(p<0.001). Horizontal lines show the mean value. Symbols on the x axis indicate the testes were too small to measure.

FIG. 8. Anti-GnRH, anti-kisspeptin and anti-GKT antibody titres in prepubertal and pubertal female rats immunised with GKT. (A) Antibody titres in prepubertal female rats. (B) Antibody titres in pubertal female rats. Animals received a single immunisation on day 0. Titres were measured at day 60. For the comparison of the titres a non-parametric test of Kruskal Wallis and a multiple comparison of Dunn were used. Horizontal bars show means. Significant differences from placebo are indicated; *(p<0.05); **(p<0.01) and ***(p<0.001). Horizontal lines show the mean value.

FIG. 9. Estradiol (E2) levels in prepubertal female rats immunised once on day 0 with GKT. (A) placebo females (B) immunised females. Animals received a single immunisation on day 0 and no further immunisation up to 300 days of monitoring (C) Box and whiskers graph representation of E2 mean values of placebo and immunised animals. For comparison, a two-way ANOVA and a Student-Newman-Keuls post hoc test were used. Statistical differences between groups at different times are indicated. *(p<0.05); **(p<0.01).

FIG. 10. Estradiol (E2) levels in pubertal female rats immunised once on day 0 with GKT. (A) placebo animals. (B) immunised animals. Animals received a single immunisation on day 0 and no further immunisation up to 300 days of monitoring (C) Box and whiskers graph representation of E2 mean values of placebo and immunised animals. For comparison a two-way ANOVA and a Student-Newman-Keulspost hoc test were used. Statistical differences between groups at different times are indicated. *(p<0.05); **(p<0.01).

### EXAMPLES AND EXPERIMENTS

### Materials and Methods

### Animals

Copenhagen male and female rats were purchased from the Center for Laboratory Animal Production (CENPALAB), Havana, Cuba and maintained in the animal house at the Center for Genetic Engineering and Biotechnology (CIGB) at Camagüey, Cuba. The animals were kept in a controlled environment at 20° C., humidity 65%, and photoperiod 14 h light and 10 h dark. Water and sterilised feed was available ad libitum.

The experiments utilised rats of different ages as follows: adult males were 10-12 weeks of age, pubertal male and female rats were 6-8 weeks of age and prepubertal male and female rats were 3-4 weeks of age at the commencement of the experiments. These different ages were used to determine whether differing responses were elicited at different stages of development.

All the experiments were approved by the Havana ethical committee of the CIGB in accordance with the Cuban and international animal care guidelines for use of laboratory animals.

### Immunogen Preparation and Animal Immunisations

The peptide immunogen combined the kisspeptin-10 peptide sequence linked to the GnRH peptide sequence through a hepatitis B T helper peptide sequence (designated GKT). The sequence Ac-Y-N-W-N-S-F-G-L-R-F-G-F-F-L-L-T-R-I-L-T-I-P-Q-S-L-D-G-E-H-W-S-Y-G-L-R-P-G-NH2 was custom synthesised by EZ bio labs (Parsippany, NJ 07054, USA) and purified to >98% (HPLC). The NH2 terminus was acetylated to decrease degradation by amino peptidases and the carboxyl terminal sequence amidated to reduce degradation by carboxyl peptidases.

For the vaccine emulsion preparation, the lyophilized GKT-peptide (750 μg) was suspended in 250 μL of phosphate buffered saline (PBS 10 mM, pH 7.4) and 250 μL of Montanide ISA 51 VG adjuvant (Seppic, France) followed by mixing at 3500 rpm for 30 minutes. The resulting emulsion was administered by subcutaneous (sc) injection at four points along the back of the rats. Placebo for control rats was prepared identically but without GKT-peptide.

### Experiment 1: Immunisation of Adult Male Rats

Five adult male rats were immunised with GKT as described above four times at fortnightly intervals from day 0 to establish efficacy of the immunisation before attempting studies with a single immunisation in pubertal and prepubertal rats. Five adult male rats (controls) received the same immunisation but lacking GKT. Blood sampling for titre estimation and hormone analysis, and testis length monitoring intervals were as described below in the results. After 300 days the animals were euthanised. Continuous monitoring of the same rats was opted for rather than killing rats at regular intervals (which would have required over 500 rats) to minimise use of animals and cost saving.

### Experiments 2 and 3: Immunisation of Prepubertal and Pubertal Male and Female Rats

Ten male and ten female rats of 3-4 weeks of age (prepubertal) and ten male and ten female rats of 6-8 weeks of age (pubertal) were immunised at day 0 as described in experiment 1. Five additional animals for each group served as controls (placebo) that received the same immunisation procedure but lacking GKT. Due to partial recovery of testis size in immunised males after about 150 days, the male rats received a second identical immunisation at 180 days. The female rats did not receive this second immunisation as estradiol levels remained low in contrast to the males in which testosterone levels recovered at day 150 after immunisation. Collection of blood samples (serum), monitoring of testis length, testosterone and estradiol levels continued to 300 days (see results for sampling intervals), at which stage they were euthanised. Experiment 2 was designated as the male rat experiment and experiment 3 as the female rat experiment.

### ELISA Assays for Kisspeptin, GnRH and GKT Antisera Titre Determinations

The concentrations of circulating anti-GnRH, anti kisspeptin and anti GKT antibodies were determined by an indirect Enzyme Linked Immunosorbent assay (ELISA). The solid-phase ELISA was performed using 96-well polystyrene plates (high binding, Nunck), coated with 10 μg/ml of Kisspeptin, or GnRH or GKT and incubated overnight at 4° C. Plates were then incubated in phosphate buffered saline (PBS) (pH 7.4), bovine serum albumin (BSA) (Sigma-Aldrich, USA) 2% v/v solution, for 60 min at 37° C. The plates were washed three times with PBS-Tween 20 (0.05%). Diluted serum samples were then added and incubated for 3 h at 37° C. Following three PBS-Tween 20 (0.05%) washes, rat anti-IgG conjugated to peroxidase was added ( 1/8000) and the plates were incubated for 60 min at 37° C. Orthophenyliendamine (OPD) chromogen and the H₂O₂ substrate dissolved in buffer (dibasic sodium phosphate 0.02 M, pH 5) was added and incubated for 20 min at room temperature. The reaction was terminated by the addition of H₂SO₄, 2.5 N. The plates were read at 492 nm with the microtiter plate reader (Multiscan, Labystem, Finland).

### Testosterone Determination

Testosterone levels were determined using the commercial TESTO CT2 kit, (CIS Bio International, France). The sensitivity of the method, defined as the detectable concentration equivalent to twice the standard deviation of the zero-binding value, was approximately 0.1 nmol/L. The cross reactivity of the assay against naturally occurring steroids was less than 1%. Serum samples and standards of 25 μl were added directly to the pre-coated tubes incubated for 1 h at 37° C., washed with distilled water and read in a gamma counter.

### Estradiol Determination

Estradiol (E2) determination used the Mouse/Rat Estradiol ELISA kit Ab108667, Abcam, USA. In brief; anti-E2 antibody coated wells were incubated with duplicate E2 standards, controls, samples, and E2 peroxidase conjugate at room temperature for 120 min. Unbound E2 peroxidase conjugate was then removed and the wells were washed. A solution of TMB Reagent was then added and incubated at room temperature for 15 min. The colour development was stopped with the addition of “Stop Solution”, and the absorbance was measured spectrophotometrically at 450 nm. The sensitivity was 8.68 pg/ml (kit manufacturer).

The estradiol (E2) determination was carried out at the Vaccine department of CIGB, where non radioactive assays were performed as known in the art. The Mouse/Rat Estradiol ELISA kit Ab108667, Abcam, US was employed. In brief; anti-E2 antibody coated wells were incubated with duplicate E2 standards, controls, samples, and E2 peroxidase conjugate at room temperature for 120 min. Unbound E2 peroxidase conjugate was then removed and the wells were washed. A solution of TMB reagent (3,3′,5,5′-Tetramethylbenzidine) was then added and incubated at room temperature for 15 min. The colour development was stopped with the addition of “Stop Solution”, and the absorbance was measured spectrophotometrically at 450 nm. The sensitivity for this kit is 8.68 pg/ml and the intra and inter assays reported is ≤9 and ≤10, respectively. The linearity respond to a curve of r2=0.99872. All the experimentation and data processing was carried out according the GMP guidelines for animal experimentation and sample processing. Where needed the samples were diluted in phosphate buffered saline (PBS) (pH 7.4), bovine serum albumin (BSA) (Sigma-262Aldrich, USA) 2% v/v solution.

### Fertility Monitoring

The first mating of the rats with fertile counterparts was carried out in all animals 90 days after commencement of the experiments. A second mating was performed for experiment 1 animals at 150 days. In experiments 2 and 3, prepubertal and pubertal male and female rats were mated with fertile counterparts (of proven fertility) at 90,150 and 250 days after the start of the experiment. The third mating was done after carrying out a booster immunisation on day 180 in the males but not in the females who were immunised only once at day 0. Immunised females were paired with normal fertile males (of proven fertility) and immunised males were paired with fertile females (of proven fertility). The pairs (one per cage) remained together for 2 weeks and cages were examined every day for offspring and numbers recorded.

For mating, one male and one female were housed together in a cage of 212 square inches. Nesting material was provided in the cage. The reproductive capacity of their untreated partners (female and male, as appropriate) was checked in each case through their previous mating with healthy adult rats. When the female was noticeably pregnant through the observation of a vaginal plug, the male was removed from the cage.

### Measurement of Testes in Males.

The length of testes of the males in all groups were measured using a vernier caliper at the beginning of the experiment, at 60 days, and thereafter approximately every 30 days until the culmination of the experiment at 300 days.

### Statistical Analysis.

In order to check whether the data complied with a normal distribution, the Kolmogorov-Smirnov test was performed. For fertility analysis, according to the not normal distribution of the data, we used a non parametric Chi square test and designating (p<0.05) as significant when the Chi SqueredSquared test was >3.84 and (p<0.01) when this value was >6.63. For the study of testosterone and estradiol concentrations as well as the evaluation of the size of the testes at different times, a bifactorial ANOVA followed by a Dunn's multiple rank test was used. When the data did not meet the homogeneity of variance, the non-parametric Kruskal Wallis test was employed. All data processing was carried out using the statistical package Prism Graph Pad. Version 6.0 (StatSoft, Inc).

### Results

### Experiment 1: Immunisation of Adult Male Rats

### Anti-GnRH, Anti Kisspeptin and Anti GKT in Adult Male Rats.

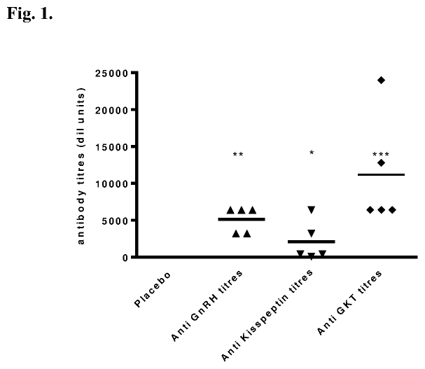

In the first experiment, as a positive control, the rats were immunised four times fortnightly as in previous studies with a GnRH immunogen preparatory to conducting studies with a single immunisation which is desirable for the objective of developing practical immunocontraception in livestock and companion animals. The four immunisations were performed with 750 ug of the GKT peptide adjuvanted in Montanide ISA 51 VG, which has previously been used in a GnRH vaccine in male rats [26, 27]. When tested at 60 days the highest antibody titres were induced against the GKT peptide (mean 1:10000 p<0.001) (FIG. 1). The next highest titre was the anti-GnRH response (mean 1: 5000) (p<0.01) followed by the anti-kisspeptin response, 1: 2500 (p<0.05). (The same order of titres was observed in the experiment in pubertal males but prepubertal males had highest titres against GnRH-see below).

Testosterone Levels in Adult Male Rats Immunised with GKT Peptide.

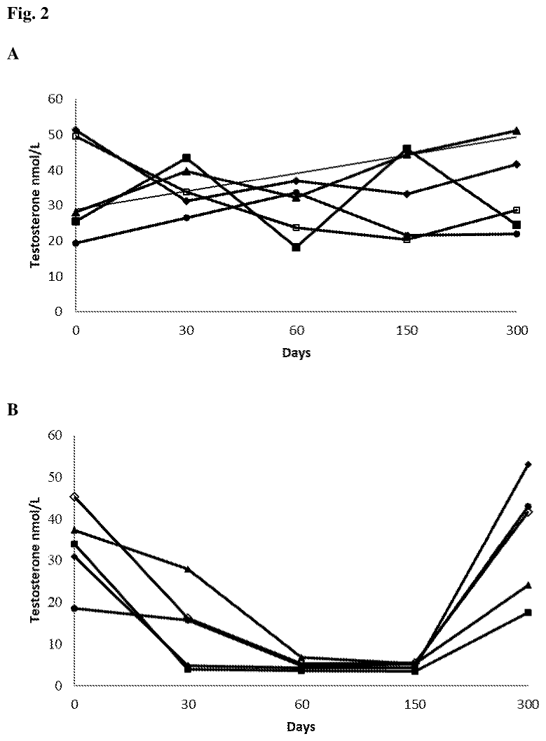

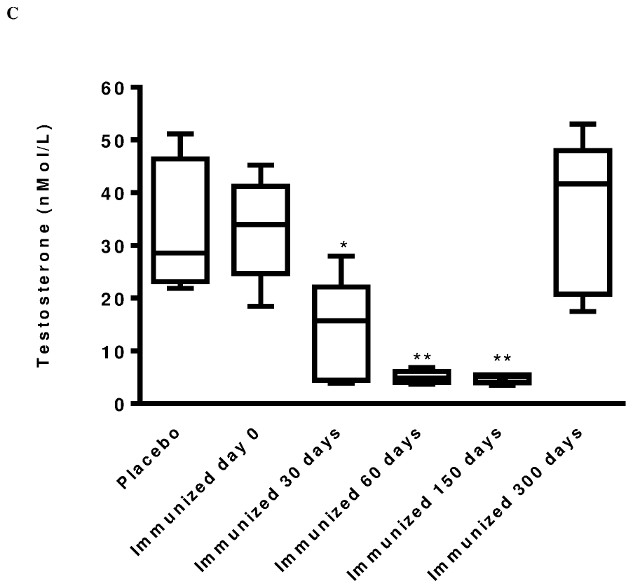

Testosterone levels at the commencement of the experiment were between 20 and 50 nmol/L which is within the normal range for adult male rats (FIG. 2A). Sixty days after the start of the study testosterone levels in immunised males had declined to near undetectable levels (5 nmol/L) (FIG. 2B) and remained so until day 150. Thereafter testosterone levels increased and by day 300 the levels had recovered to almost the starting levels. FIG. 2C shows that testosterone levels were significantly decreased in immunised animals at days 30, 30 (P<0.05) and at days 60 and 150 (P<0.001) in relation to day 0 of the experiment utilising a two-way ANOVA and a Student-Newman-Keuls post hoc test. Control animals (placebo) maintained normal levels throughout the study up until 300 days when the experiment was terminated.

Testes Length in Adult Male Rats Immunised with GKT

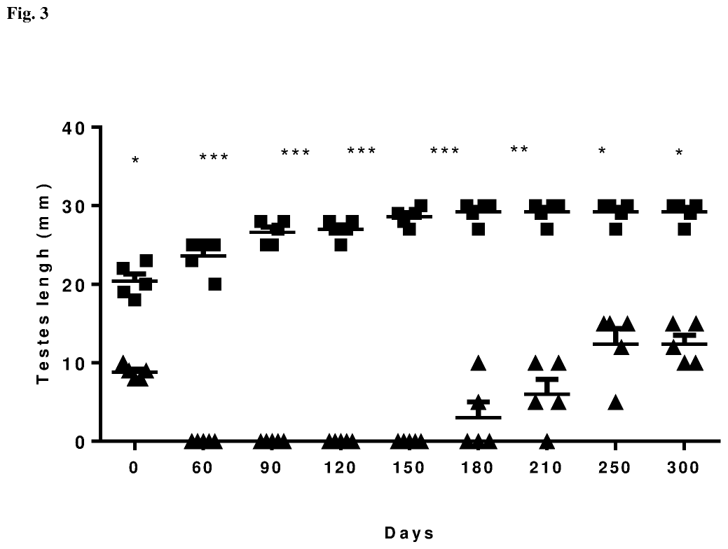

Testis length in immunised males exhibited a dramatic reduction to almost unmeasurable length by day 60 after immunisation (FIG. 3). They remained at this size until day 150 whereafter they increased in size up to day 250 and then remained at the same length until day 300 (P<0.05). For comparison, a two-way ANOVA and a Student-Newman-Keuls post hoc test was used Interestingly, the testes only increased to 40-50% of control animals and remained significantly smaller than in control animals and did not regain the testes length of control animals for the duration of the experiment up to 300 days.

Mating Outcomes of Adult Rats Immunised with GKT Peptide.

In order to determine the effect of GKT immunisation of adult male rats on fertility they were paired with fertile female rats on day 90 and day 150. On day 90 only one of five male rats produced offspring and on day 150 two males sired litters (Table 1). The immunised rat which fathered a litter at day 90 had higher testosterone levels than the infertile rats and at day 150 the two fertile male rats had higher testosterone levels than the infertile rats. All of the placebo immunised rats produced litters. The differences in fertile/infertile male rats between immunised and placebo animals were significant by Chi square analysis (p<0.05).

TABLE 1
Outcome of mating GKT and placebo immunised
adult males with fertile female rats

| | GKT immunised | | Placebo immunised | |
|---|---|---|---|---|
| Days after immunisation | 90 | 150 | 90 | 150 |
| Number of males siring litters * | 1/5 | 2/5 | 4/5 | 5/5 |

* indicates number of males siring litters out of total males

### Experiment 2: Immunisation of Prepubertal and Pubertal Male Rats

Having established that GKT was efficacious in inhibiting fertility after four immunisations in adult male rats, the potential for a single immunisation with GKT during development to inhibit reproduction in prepubertal and pubertal male and female rats was examined.

### Titres of Anti GnRH, Kisspeptin and GKT Immunogens in Prepubertal and Pubertal Males

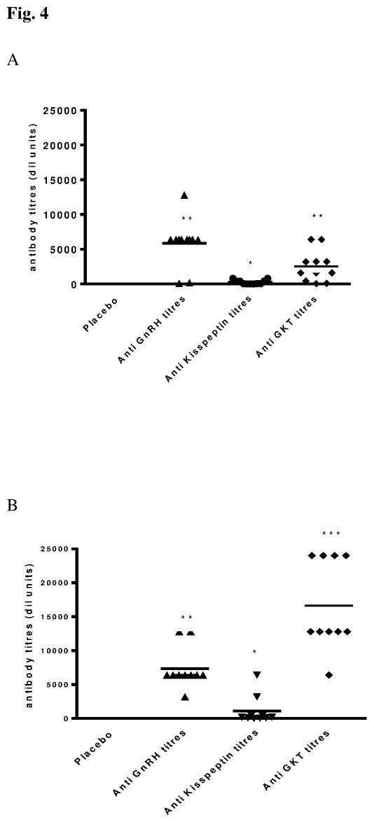

All prepubertal males (n=10) and pubertal males (n=10), developed anti-GnRH, anti-kisspeptin and anti-GKT antibodies after a single immunisation albeit some at low titres (FIG. 4). In the prepubertal males highest antibody titres were developed against GnRH˜1: 6000 (p<0.01), followed by GKT˜1: 3000 (p<0.01) and then followed by kisspeptin where titres only reached an average of around ˜1:500 (p<0.05) (FIG. 4A). On the other hand, in the pubertal males, a higher antibody titre was obtained against GKT, ˜1:18000 (p<0.001), followed by the anti-GnRH titre ˜1:8000 (p<0.01) and the anti-kisspeptin titre ˜1:1000 (p<0.05) (FIG. 4 B). The higher titres achieved in pubertal compared to prepubertal rats may be related to the greater maturity of the immune system of pubertal compared to prepubertal animals. (similar results were obtained in pubertal and prepubertal female rats-see below). The interesting difference of highest titre against GnRH in prepubertal males but highest titre against GKT in pubertal males is noted.

Testosterone Levels in Prepubertal and Pubertal Male Rats Immunised with GKT Peptide

### Prepubertal Males

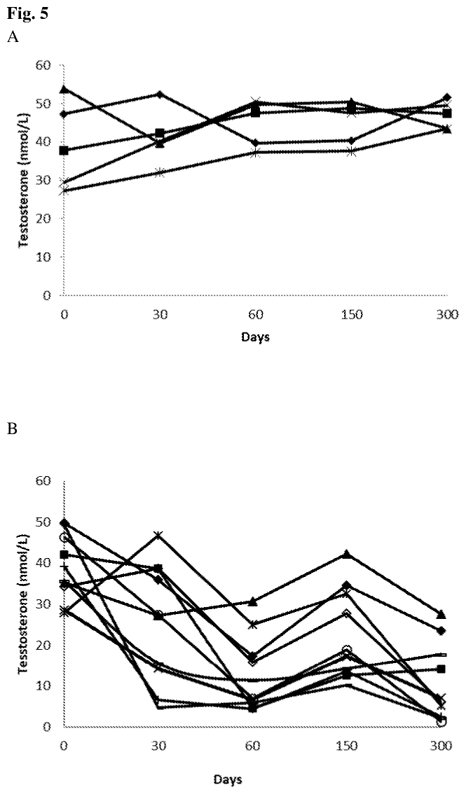

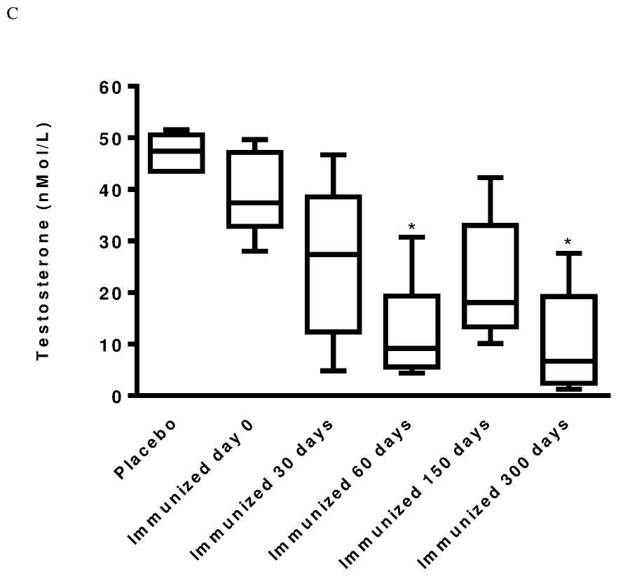

Testosterone levels in prepubertal males (4-5 weeks of age) were between 28 and 55 nmol/L in the controls and the immunised animals at the start of the experiments. By day 60 all immunised animals showed a decrease in testosterone levels to between 5 and 30 nmol/L (FIG. 5 B). These levels increased thereafter at day 150. The good responders reached levels between 10 and 18 nmol/L, which were still significantly lower than the placebo control (P<0.05), while the poor responders reached normal levels between 28 and 42 nmol/L.

In view of this recovery it was determined whether re-immunisation would restore inhibition of reproduction. At day 180, the same dose of immunogen was administered as at the beginning of the experiment. A significant reduction of testosterone levels was obtained in most of the animals (2-17 nmol/L) (p<0.05), however, 2 animals remained poor responders until the end of the experiment. Mean values are depicted in the box and whisker plots which show that there were significant decreases in testosterone in immunised animals at day 60 and 300 after reimmunisation (FIG. 5 C).

### Pubertal Males

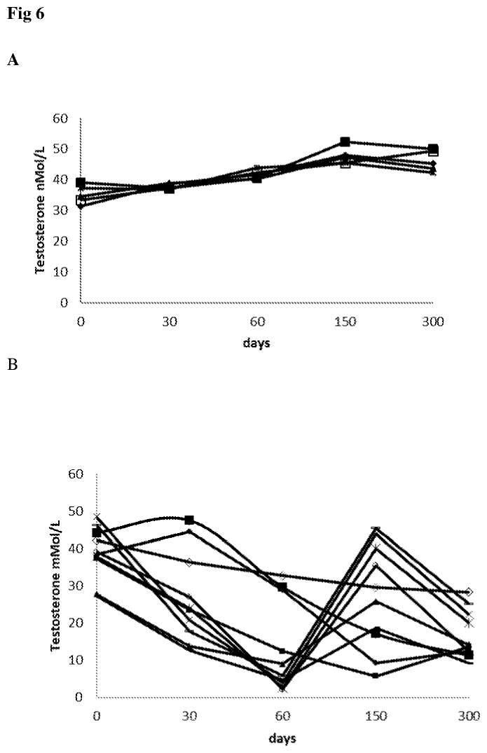

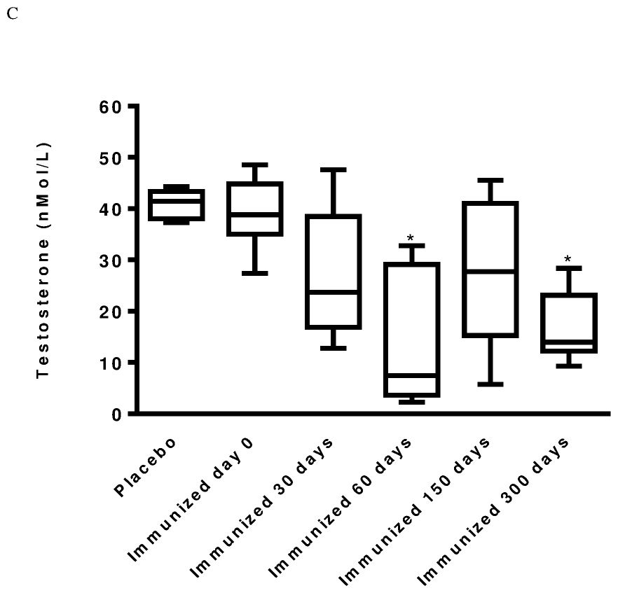

The testosterone levels in both immunised and placebo pubertal males at the start of the experiment were (30-50 nmol/L) (FIG. 6 A and B). All immunised males showed a decline in testosterone levels by 60 days. Most immunised males then showed an increase in testosterone by 150 days. Interestingly, two of the males showed a delayed decline at 150 days reflecting variation in response. In view of the ensuing increase in testosterone in most males a second immunisation was administered at 180 days and all except the two delay-response males showed a decline in testosterone (FIG. 6 A). Mean values are depicted in the box and whisker plot which shows that there were significant decreases in testosterone in immunised animals at days 60 and 300 after reimmunisation on day 180 (FIG. 6 C).

Testes Length in Prepubertal and Pubertal Male Rats Immunised with GKT Peptide.

### Prepubertal Males

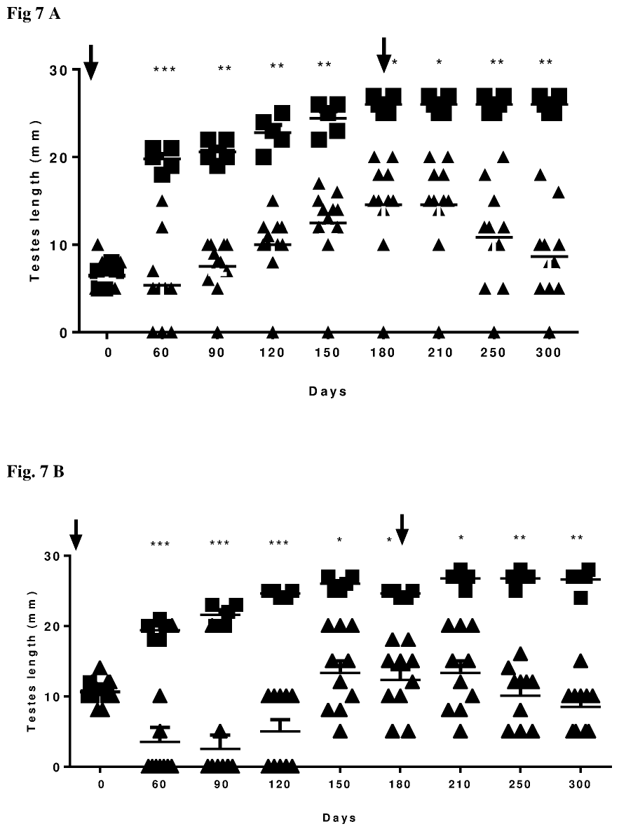

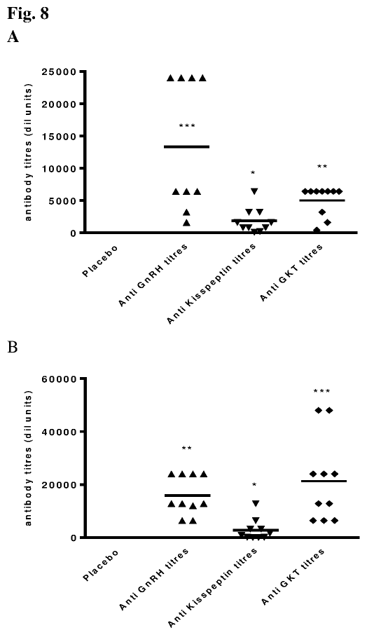

Placebo prepubertal rats exhibited a steady growth of testicular length from 60 days until about 180 days when they reached a plateau (FIG. 7A). Testes in all immunised males did not increase in length until day 120 and were significantly shorter than that of control animals. Thereafter, testes increased in length in immunised animals to reach a plateau at about 50% of controls. Booster immunisation at 180 days then induced a decline in testis length up to 300 days when they approached the same size as initially at day 0 (FIG. 7A). The increase in the length of the testes corresponded to the increase in testosterone levels at 150 days.

### Pubertal Males

Placebo pubertal males, as expected, had slightly bigger testes than prepubertal males at the start of the experiment. They increased in length to reach a maximum at day 120 (FIG. 7 B). Immunised males exhibited a much more robust decline in testes length at day 60, compared with immunised prepubertal animals, to reach almost unmeasurable size which was maintained until day 90 (FIG. 7 B). At day 120, in half of the males the testes remained small but the other half showed recrudescence. Thereafter the testes increased in length to reach a plateau of 50% of placebo animals as occurred with the immunised prepubertal animals at 150 and 180 days and then declined somewhat after the booster immunisation.

Mating Outcomes of Prepubertal and Pubertal Male Rats Immunised with the GKT Peptide.

### Prepubertal Males

All placebo males sired litters (Table 2). In GKT immunised males 90% were infertile when mated at 90 days, 80% infertile when mated at 150 days and 80% infertile when mated at 250 days. The immunised male which was fertile at the first mating remained fertile in the subsequent matings, even after reimmunisation at 180 days, indicating that individual animals can be resistant to immunisation. The titres for GKT, GnRH and kisspeptin for this animal were 1:1600, 1:6400 and 1:50, respectively.

TABLE 2
Outcome of mating GKT and placebo immunised
pre-pubertal males with fertile female rats

| | GKT | | | Placebo | | |
|---|---|---|---|---|---|---|
| | immunised | | | immunised | | |
| Days after immunisation | 90 | 150 | 250 | 90 | 150 | 250 |
| Number of males siring litters * | 1/10 | 2/10 | 2/10 | 5/5 | 5/5 | 5/5 |

* indicates number of males siring litters out of total males

### Pubertal Males

Similar results were obtained in studies on immunisation of pubertal males. All placebo males sired litters (Table 3). For GKT immunised males, 80% were infertile when mated at 90 days, 70% infertile when mated at 150 days and 80% infertile when mated at 250 days. A male which was fertile at the first two matings was infertile on the third mating after reimmunisation at 180 days, indicating that individual animals can initially be resistant to immunisation. The titres for GKT, GnRH and kisspeptin antibodies in this animal at day 45 were 1:12800, 1:6400 and 1:3200, respectively.

TABLE 3
Outcome of mating GKT and placebo immunised
pubertal males with fertile female rats

| | GKT | | | Placebo | | |
|---|---|---|---|---|---|---|
| | immunised | | | immunised | | |
| Days after immunisation | 90 | 150 | 250 | 90 | 150 | 250 |
| Number of males siring litters * | 2/10 | 3/10 | 2/10 | 5/5 | 5/5 | 5/5 |

* indicates number of males siring litters out of total males

### Experiment 3: Immunisation of Pubertal and Prepubertal Female Rats

### Titres to GnRH, Kisspeptin and GKT Immunogens

All prepubertal females (n=10) and pubertal females (n=10), developed anti-GnRH, anti-kisspeptin and anti-GKT antibodies after a single immunisation (FIG. 8). As in prepubertal males, prepubertal females had the highest titers against GnRH˜1:14000 (p<0.001), followed by against GKT˜1:5000 (p<0.01) and lowest against kisspeptin ˜1:2500 (p<0.05). In contrast, and as for pubertal males, the pubertal females had a higher antibody titre against GKT, ˜1:23000 (p<0.001), followed by the anti-GnRH titration ˜1:17000 (p<0.01) and lowest against kisspeptin ˜1:3000 (p<0.05).

Thus, there is a clear difference in antibody production in relation to development. In prepubertal males and females highest titres were against GnRH while in pubertal males and females highest titres were against GKT.

Estradiol Levels in Prepubertal and Pubertal Female Rats Immunised with GKT Peptide

### Prepubertal Females

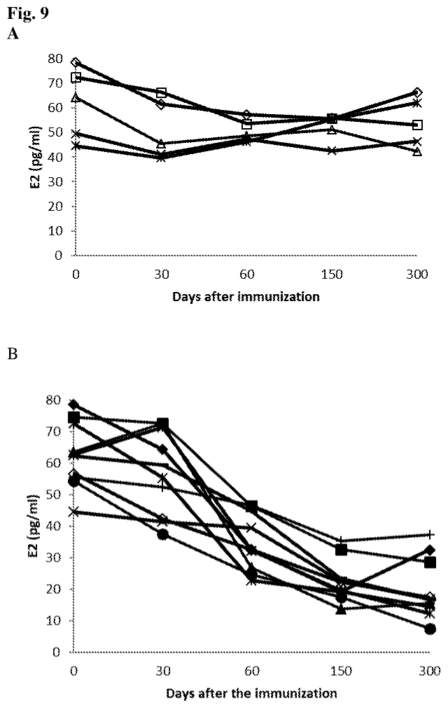

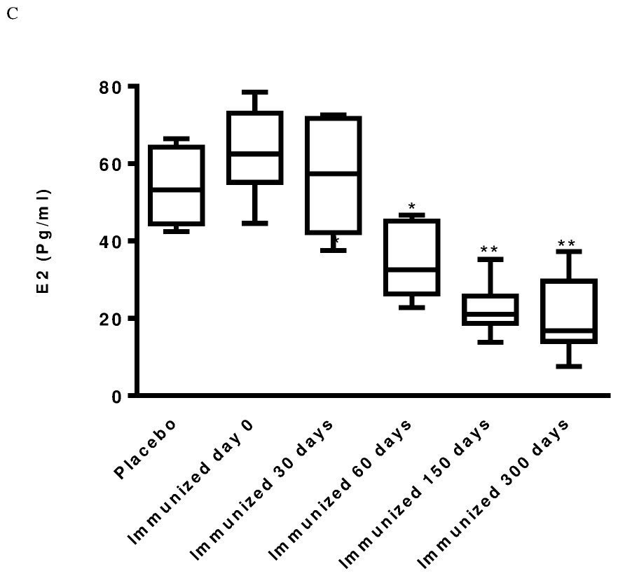

At the start of the study estradiol values were between 45 and 80 pg/ml in prepubertal placebo and GKT groups. While the estradiol levels remained at this level in the placebo females, there was a continuing decline in the GKT immunised females to 25-50 pg/ml at day 60; while it ranged between 10 and 30 pg/ml at day 150 (p<0.05) and between 10 and 30 pg/ml on days 300 (p<0.05) in the best responders (FIG. 9 A and B). Mean values are depicted in the box and whisker plots which show that there were significant decreases in estradiol in immunised animals at days 60, 150 and 300 (FIG. 9 C).

### Pubertal Females

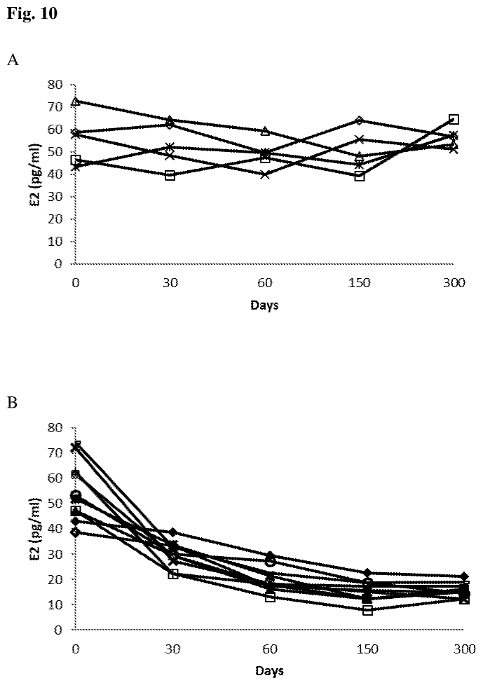

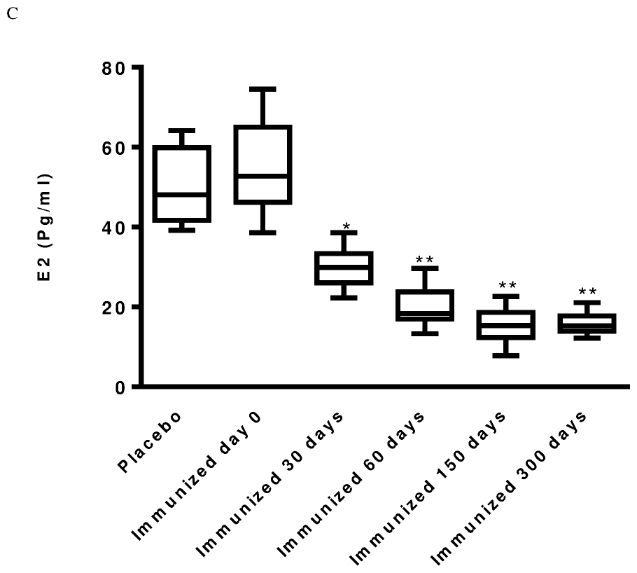

At the start of the study estradiol levels in placebo and immunised groups were 40-75 pg/ml and remained at this level in control (placebo) animals (FIG. 10A). The immunised females exhibited a robust decline in estradiol levels by day 30 (p<0.05) which continued at 60 days to reach low levels of 10-25 pg/ml on days 150 and 300 (FIG. 10 B). The steroid levels were more homogeneous in this group than the prepubertal group. Mean values are depicted in the box and whisker plot which shows that there were significant decreases in estradiol in immunised animals at days 30, 60, 150 and 300 (FIG. 10 C).

Mating of Prepubertal and Pubertal Female Rats Immunised with GKT Peptide.

### Prepubertal Females

The placebo females all produced offspring at all three matings. Prepubertal immunised females showed reduced fertility at the first mating at 90 days (80% infertile), second mating at 150 days (70% infertile) and third mating at 250 days (70% infertile) (Table 4). These differences were all significantly different from placebo. The GKT immunised rats that produced litters, exhibited similar anti GKT, anti GnRH and anti Kisspeptin antibody levels.

TABLE 4
Outcome of mating GKT and placebo immunised
pre-pubertal females with fertile male rats

| | GKT | | | Placebo | | |
|---|---|---|---|---|---|---|
| | immunised | | | immunised | | |
| Days after immunisation | 90 | 150 | 250 | 90 | 150 | 250 |
| Number of females having | 2/10 | 3/10 | 3/10 | 5/5 | 5/5 | 5/5 |
| litters * | | | | | | |

* indicates number of females producing litters out of total females

### Pubertal Females

All but one (first mating) pubertal placebo females produced offspring in all three matings (Table 5). Impressively, all GKT immunised pubertal females had no offspring from all three matings at days 90, 150 and 250 in accordance with the low estradiol levels in these animals. These result correspond with the generally higher antibody titres generated in pubertal females.

TABLE 5
Outcome of mating GKT and placebo immunised
pubertal females with fertile male rats

| | GKT | | | Placebo | | |
|---|---|---|---|---|---|---|
| | immunised | | | immunised | | |
| Days after immunisation | 90 | 150 | 250 | 90 | 150 | 250 |
| Number of females having | 0/10 | 0/10 | 0/10 | 4/5 | 5/5 | 5/5 |
| litters * | | | | | | |

* indicates number of females producing litters out of total females

### DISCUSSION

GnRH vaccines have been widely employed for immunosterilisation [3-12, 28-30]. A number (the majority) of these require at least two vaccinations and fail to convey infertility to 100% of immunised animals. Furthermore, there is a recovery of fertility over time, thus requiring reimmunisation [8-11]. As GnRH secretion is stimulated by kisspeptin [14-24, 31-35] in all mammalian species examined to date, it is posited that a combination of GnRH and its upstream regulator, kisspeptin, connected by a hepatitis B peptide sequence within a single molecule would be a more efficacious immunogen than a single peptide immunogen targeting only one level in the hypothalamic-pituitary-gonadal axis. It is further considered that permanent sterilisation is achieved by immunising at particular windows of development as was achieved in studies on neonatal immunisation with GnRH conjugate in sheep [13]. We also examined whether a single immunisation at a specific stage of development might be sufficient to elicit immunosterilisation.

All male and female rats immunised with GKT produced antibodies against GnRH, kisspeptin-10 and the GKT immunogen supporting the notion that two levels of the hypothalamic-pituitary axis can be targeted successfully with a single immunogen. The highest titres were against GKT and GnRH. Prepubertal immunisation of males and females yielded highest titres against GnRH. In contrast pubertal immunisation of males and females and adult males yielded highest titres against GKT suggesting that stage of development influences the titres to the different peptides. In all instances titres against kisspeptin-10 were lowest. This may reflect the endogenous status of GnRH and kisspeptin neurone development and secretion and its interplay with GKT immunisation. Whatever the explanation, it is likely that the ensuing infertility in the males and females arises from a combination of immunoneutralisation of GnRH and kisspeptin-10. Both are small molecules (ten amino acids) such that antibodies raised to any epitope within the decapeptide sequence is likely to prevent receptor binding by obscuring ligand amino acids involved in binding to cognate receptors and/or by antibody binding to amino acids not directly involved in binding the receptors but creating steric hindrance. This is supported by the reports on GnRH immunogens [8-11, 26]. Indeed, it has been previously shown that antibodies to various epitopes in GnRH prevent activation of the GnRH receptor [26, 29]. An antibody binding site constitutes about six amino acids such that the array of antibodies directed at the amino terminal, carboxy terminal and central amino acids which have been reported would all impair receptor binding through pGlu¹ of GnRH interaction with the receptor Asn²¹², His² of GnRH interaction with receptor Lys¹²¹/Asp⁹⁸, Tyr⁵ of GnRH with receptor Tyr²⁹⁰, Arg⁸ of GnRH with receptor Asp³⁰², Pro⁹/Gly¹⁰ of GnRH with Trp¹⁰¹/Asp¹⁰² [33, 37-39]. A similar situation is likely to pertain to kisspeptin antibodies but this has not been examined in this or other studies. Interestingly, the lowest titres of antisera were against kisspeptin-10. This may suggest that the kisspeptin peptide sequence has less “attractive” epitopes for antibody production. Nevertheless, the titres achieved along with GnRH antibodies are likely to have contributed to the infertility realised.

Adult male rats immunised four times against GKT at two weekly intervals produced antibodies to GnRH, kisspeptin-10 and GKT and had a marked reduction in testis length, thus establishing the efficacy of GKT as an immunogen. By day 60 testicular length was unmeasurable and remained at this level until day 180 when testis length began to increase and plateaued out at 40-50% of controls at day 250 and day 300. Testosterone levels also increased at this stage but unlike testis length returned to normal levels. This suggests that Leydig cell function returns to normal but spermatogonia and Sertoli cells do not. It is possible that differential LH and FSH inhibition by the immunogen may play a part but these hormones were not measured in the current study. Unfortunately, the testes in these animals and the subsequent studies were not fixed for histological study of Leydig cell, spermatogonia and Sertoli cells. It is intriguing that although testis length recovered to only 40-50% of controls two of the five males recovered fertility.

This study was followed up by examining if a single immunisation with GKT in prepubertal and pubertal males could render them infertile. All immunised animals produced antibodies to GnRH, kisspeptin and the immunogen GKT with highest titres in prepubertal males against GnRH and in pubertal males against GKT (see discussion above). Both immunised groups showed a marked decline in testosterone levels up to day 60 and then a recovery to normal levels by day 150. There were individual differences, however, and about half of prepubertal and pubertal animals did not exhibit a recovery of testosterone to normal levels.

Both groups had a decline in testis length to day 90 after the single immunisation. The pubertal group decline in testis length was similar to that shown in the adult males (almost unmeasurable) while the prepubertal males had a much reduced decline in testis length. This result was contrary to the expectation that immunisation would be more effective in this group as it had shown irreversible infertility in rams immunised neonatally (perpubertally). It appears, therefore to be a species difference. The greater effect of immunisation on testis length in the pubertal group compared to the prepubertal group was paralleled by 80-90% infertility in the pubertal group compared with 70-80% infertility in the prepubertal group. The bottom line is that a single immunisation with GKT elicited antibody production in all animals and infertility in a high percentage. However, this fell short of the aim to utilise only one immunisation to be 100% efficacious and induce permanent infertility as testis size and testosterone recovered. Nevertheless, GKT appeared to be more effective than some GnRH vaccines studied [8-11, 26].

The most impressive results were realised in pubertal and prepubertal female rats in which a single GKT immunisation led to a decrease in estradiol which continued up until 300 days with no indication of recovery. The main end point for all of the studies was the inability to produce offspring. Twenty to thirty percent of prepubertal females were fertile in spite of low estradiol levels. Impressively, all immunised pubertal females were infertile when tested at 90,150 and 250 days after a single immunisation. Unfortunately, ovaries were not collected but the low estradiol indicates that there was an absence of follicular development. This result in pubertal females therefore satisfies our objective of a single immunisation resulting in apparent permanent sterilisation (at least to 300 days). Future studies will attempt to mimic this approach and confirm similar findings in other species such as dogs, cats, livestock and wildlife in which a single immunisation and permanent sterilisation is desirable.

The present invention undertakes a novel approach to indicate that the dual GKT immunogen is more efficacious and achieves sterilisation after a single immunisation. To that end, the demonstration of sterilisation of male and female rats after a single vaccination, and in particular 100% sterilisation of pubertal females for at least 300 days occurred. In contrast numerous GnRH vaccines have all required at least two vaccinations and follow up vaccinations. This demonstrates that the dual immunogen of GnRH and kisspeptin is more efficacious.

The findings clearly demonstrate that a dual immunogen comprising GnRH and kisspeptin-10 is efficacious in generating immunoneutralising antibodies and infertility. The findings also indicate that the responses to GKT is sex and development dependent; thereby setting the scene for further exploration in using GKT in target animal species. The encouraging total infertility for at least 300 days after a single immunisation with GKT in pubertal female rats suggest that this may be achievable in other species.

### Acknowledgements

The Technology Innovation Agency of South Africa supported the research through a grant to RPM. The Centre for Genetic Engineering and Biotechnology of Camaguey in Cuba supported the animal studies. We thank Ayni Rodriguez, Andres Serradelo and Hilda Garay for the experimental contribution to this work.

The following references are included herein by reference.

### REFERENCES

1. Kirkpatrick, J. F. and A. Turner, Achieving population goals in a long-lived wildlife species (Equus caballus) with contraception. Wildlife Research, 2008. 35(6): p. 513.
2. Bertschinger, H. J., et al. Porcine zona pellucida immunocontraception of African elephants (Loxodonta africana): beyond the experimental stage. in IVth International Wildlife Management Congress. 2012. Durban, South Africa. P. 95-102
3. Bertschinger, H. J. and E. S. Sills, Contraceptive Applications of GnRH—analogs and Vaccines for Wildlife Mammals of Southern Africa: Current Experience and Future Challenges, in Gonadotropin-releasing hormone (GnRH). Production, structure and function. E. S. Sills, Editor. 2013 Nova Science Publishers Inc: New York. p. 85-107.
4. Elhay, M., et al., Suppression of behavioural and physiological oestrus in the mare by vaccination against GnRH. Australian Veterinary Journal, 2007. 85(1-2): p. 39-45.
5. Janett, F., et al., Suppression of testicular function and sexual behavior by vaccination against GnRH (Equity™) in the adult stallion. Animal Reproduction Science, 2009. 115(1-4): p. 88-102.
6. De Nys, H. M., et al., Vaccination against GnRH may suppress aggressive behaviour and musth in African elephant (Loxodonta africana) bulls—a pilot study. Journal of the South African Veterinary Association, 2010. 81(1); p. 8-15
7. Janett, F., et al., Vaccination against gonadotropin-releasing factor (GnRF) with Bopriva significantly decreases testicular development, serum testosterone levels and physical activity in pubertal bulls. Theriogenology, 2012. 78(1): p. 182-188.
8. Dalin, A. M., O. Andresen, and L. Malmgren, Immunisation against GnRH in Mature Mares: Antibody Titres, Ovarian Function, Hormonal Levels and Oestrous Behaviour. Journal of Veterinary Medicine Series A, 2002. 49(3): p. 125-131.
9. Botha, A. E., et al., The use of a GnRH vaccine to suppress mare ovarian activity in a large group of mares under field conditions. Wildlife Research, 2008. 35(6): p. 548-554.
10. Schulman, M. L., et al., Reversibility of the effects of GnRH-vaccination used to suppress reproductive function in mares. Equine Veterinary Journal, 2012. 45(1): p. 111-113.
11. Stout, T. A. E. and B. Colenbrander, Suppressing reproductive activity in horses using GnRH vaccines, antagonists or agonists. Animal Reproduction Science, 2004. 82-83: p. 633-643.
12. Turkstra, J. A., et al Effects of GnRH immunisation in sexually mature pony stallions. Anim. Reprod. Sci. 2005. 86, p. 247-259.
13. Clarke, I. J., et al., Neonatal Immunisation against Gonadotropin-Releasing Hormone (GnRH) Results in Diminished GnRH Secretion in Adulthood. Endocrinology, 1998.139(4): p. 2007-2014.
14. Plant, T. M., The role of KiSS-1 in the regulation of puberty in higher primates. European Journal of Endocrinology, 2006. 155(suppl_1): p. S11-S16.
15. Roseweir, A. K. and R. P. Millar, The role of kisspeptin in the control of gonadotrophin secretion. Human Reproduction Update, 2009. 15(2): p. 203-212.
16. Roseweir, A. K., et al., Discovery of potent kisspeptin antagonists delineate physiological mechanisms of gonadotropin regulation. The Journal of neuroscience: the official journal of the Society for Neuroscience, 2009. 29(12): p. 3920-3929.
17. Pinilla, L., et al., Kisspeptins and Reproduction: Physiological Roles and Regulatory Mechanisms. Physiological Reviews, 2012. 92(3): p. 1235-1316.
18. Harter, C. J. L., G. S. Kavanagh, and J. T. Smith, The role of kisspeptin neurons in reproduction and metabolism. Journal of Endocrinology, 2018. 238(3): p. R173-R183.
19. Scott, C. J., et al., Kisspeptin and the regulation of the reproductive axis in domestic animals. Journal of Endocrinology, 2019. 240(1): p. R1-R16.
20. Seminara, S. B., et al., The GPR54 Gene as a Regulator of Puberty. New England Journal of Medicine, 2003. 349(17): p. 1614-1627.
21. de Roux, N., et al., Hypogonadotropic hypogonadism due to loss of function of the KiSS1-derived peptide receptor GPR54. Proceedings of the National Academy of Sciences of the United States of America, 2003. 100(19): p. 10972-10976.
22. Topaloglu, A. K., et al., Inactivating KISS1 Mutation and Hypogonadotropic Hypogonadism. New England Journal of Medicine, 2012. 366(7): p. 629-635.
23. Pineda, R., et al., Critical Roles of Kisspeptins in Female Puberty and Preovulatory Gonadotropin Surges as Revealed by a Novel Antagonist. Endocrinology, 2010. 151(2): p. 722-730.
24. Kinoshita, M., et al., Involvement of Central Metastin in the Regulation of Preovulatory Luteinizing Hormone Surge and Estrous Cyclicity in Female Rats. Endocrinology, 2005. 146(10): p. 4431-4436.
25. Paltiel, H. J., et al., Testicular Volume: Comparison of Orchidometer and US Measurements in Dogs. Radiology, 2002. 222(1): p. 114-119.
26. Junco, J. A., et al., Immunotherapy of prostate cancer in a murine model using a novel GnRH based vaccine candidate. Vaccine, 2007. 25(50): p. 8460-8468.
27. Junco, J. A., et al., Prostate Cancer Immunotherapy—Strategy with a Synthetic GnRH Based Vaccine Candidate, in Advancements in Tumor Immunotherapy and Cancer Vaccines. Intech Publishers, 2012, 4, p.63-82.
28. Ferro, V. A., et al., Immunoneutralisation of GnRH-I, without cross-reactivity to GnRH-II, in the development of a highly specific anti-fertility vaccine for clinical and veterinary use. Journal of Reproductive Immunology, 2001. 51(2): p. 109-129.
29. Fromme, B., et al., A Novel Retro-Inverso Gonadotropin-Releasing Hormone (GnRH) Immunogen Elicits Antibodies That Neutralize the Activity of Native GnRH. Endocrinology, 2003. 144(7): p. 3262-3269.
30. Levy, J. K., et al., GnRH immunocontraception of male cats. Theriogenology, 2004. 62(6): p. 1116-1130.
31. George, J. T., et al., Kisspeptin-10 is a potent stimulator of LH and increases pulse frequency in men. The Journal of clinical endocrinology and metabolism, 2011. 96(8): p. E1228-E1236.
32. Fuqua, J. S., Treatment and Outcomes of Precocious Puberty: An Update. The Journal of Clinical Endocrinology & Metabolism, 2013. 98(6): p. 2198-2207.
33. Millar, R. P. and C. L. Newton, Current and future applications of GnRH, kisspeptin and neurokinin B analogues. Nature Reviews Endocrinology, 2013. 9(8): p. 451-466.
34. Albers-Wolthers, K. H., et al., Identification of a novel kisspeptin with high gonadotrophin stimulatory activity in the dog. Neuroendocrinology, 2014. 99(3-4): p. 178-89.
35. Franssen, D. and M. Tena-Sempere, The kisspeptin receptor: A key G-protein-coupled receptor in the control of the reproductive axis. Best Practice & Research Clinical Endocrinology & Metabolism, 2018. 32(2): p. 107-123.
36. Millar, R. P., et al., Region-specific antisera in molecular biology of neuropeptides: Application in quantitation, structural characterisation and metabolism of luteinizing hormone-releasing hormone, in Molecular Biology Approach to the Neurosciences, H. Soreq, Editor. 1984, Wiley and Sons: New York. p. 221-230.
37. Millar, R. P., et al., Gonadotropin-Releasing Hormone Receptors. Endocrine Reviews, 2004. 25(2): p. 235-275.
38. Millar, R. P., GnRHs and GnRH receptors. Animal Reproduction Science, 2005. 88(1-2): p. 5-28.
39. Newton, C. L., C. Riekert, and R. P. Millar, Gonadotropin-releasing hormone analog therapeutics. Minerva Ginecologica, 2018. 70(5) p. 417-515.

1. An immunogen comprising a gonadotropin releasing hormone (GnRH) peptide sequence, a kisspeptin peptide sequence and a stimulant of raising an immune response including T helper peptide sequences as in tetanus toxin, Hepatitis B and from other polypeptides.

2. The immunogen according to claim 1 wherein the T helper peptide sequence is a Hepatitis B peptide sequence.

3. The immunogen according to claim 2 wherein the Hepatitis B peptide sequence links the GnRH and the kisspeptin peptide sequences.

4. The immunogen according to any one of claim 1 to 3 wherein the immunogen is a single peptide molecule.

5. The immunogen according to claim 4 wherein the amino and carboxyl termini are extended or blocked with peptide sequences or addition of peptides or other molecules via central residues including conjugation to immunoglobulin.

6. The immunogen according to any of claim 3 to 5 wherein the order of the peptide sequences is any order, including kisspeptin, Hepatitis B and GnRH.

7. The immunogen according to any one of claim 1 to 6 having a peptide sequence having at least 80% homology to Ac-Y-N-W-N-S-F-G-L-R-F-G-F-F-L-L-T-R-I-L-T-I-P-Q-S-L-D-G-E-H-W-S-Y-G-L-R-P-G-NH2 (SEQ ID NO. 4).

8. An immunogen according to any one of claim 1 to 7 for use in a method to regulate the release of hormones in a vertebrate including modulation of reproductive hormones, to reduce fertility in a vertebrate and to treat hormone-dependent diseases including hormone-dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity, the method comprising the steps of administering the immunogen to a vertebrate in an amount effective to regulate the release of hormones in the vertebrate.

9. The immunogen for use according to claim 8 wherein the immunogen is administered to the vertebrate prepubertally, pubertally and/or as an adult.

10. The immunogen for use according to claim 9 wherein the immunogen is administered pubertally.

11. The immunogen for use according to any one of claim 8 to 10 wherein the immunogen components are singly administered.

12. The immunogen for use according to any one of claim 8 to 11 wherein the immunogen is administered by subcutaneous injection.

13. A vaccine formulation comprising an immunogen according to any one of claims 1 to 7 in an amount effective to regulate the release of hormones in a vertebrate including modulation of reproductive hormones, to reduce fertility in a vertebrate and to treat hormone-dependent diseases including hormone dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity, in combination with a pharmaceutically acceptable carrier or excipient.

14. The vaccine formulation of claim 13 wherein the peptide is conjugated to or administered with at least one carrier or adjuvant including CpGs, M59, incomplete Freund's adjuvant, complete Freund's adjuvant, alum, bile salts, vitamins, PEG, molecules which prolong half-life and attenuated toxins.

15. The vaccine formulation according to claim 13 wherein the vertebrate is a mammal selected from humans, rodents, including rats and mice, cats, dogs, livestock including cattle, horses and wildlife.

16. Use of an immunogen according to any one of claim 1 to 8 in the preparation of a medicament for use in a method to regulate the release of hormones in a vertebrate including modulation of reproductive hormones, to reduce fertility in a vertebrate and to treat hormone dependent diseases including hormone dependent tumours including prostate tumours, breast, ovary and endometrial tumours, benign hyperplasia including benign prostatic hyperplasia and uterine fibroids, endometriosis, polycystic ovarian disease, infertility, sexual dysfunction and any disorder that would benefit from an increased or decreased GnRH-dependent activity.

17. A nucleic acid which encodes the immunogen of any one of claim 1 to 7.

18. An expression vector comprising the nucleic acid of claim 17.

19. A host cell comprising the expression vector of claim 18.


SEQUENCE LISTING (4 sequences)

SEQ ID NO: 1 - protein, 10 aa, Homo sapiens
EHWSYGLRPG

SEQ ID NO: 2 - protein, 10 aa, Homo sapiens
YNWNSFGLRF

SEQ ID NO: 3 - protein, 15 aa, Hepatitus B virus
FFLLTRILTIPQSLD

SEQ ID NO: 4 - protein, 37 aa, Artificial sequence
  Immunogen sequence
YNWNSFGLRFGFFLLTRILTIPQSLDGEHWSYGLRPG